(19) United States
(12) Patent Application Publication
(10) Pub. No.: US 2020/0188911 A1
(43) Pub. Date: Jun. 18, 2020

(54) NON-OPTICAL LABEL-FREE BIOMOLECULAR DETECTION AT ELECTRIALLY DISPLACED LIQUID INTERFACES USING INTERFACIAL ELECTROKINETIC TRANSDUCTION (IET)

(71) Applicant: THE JOHNS HOPKINS UNIVERSITY, Baltimore, MD (US)

(72) Inventors: Zachary Gagnon, Baltimore, MD (US); Nicholas Mavrogiannis, Baltimore, MD (US)

(21) Appl. No.: 16/083,967

(22) PCT Filed: Mar. 13, 2017
(86) PCT No.: PCT/US2017/022005

§ 371 Date: Sep. 11, 2018

Related U.S. Application Data
(60) Provisional application No. 62/307,200, filed on Mar. 11, 2016.

(51) Int. Cl. B01L 3/00; G01N 27/02; G01N 27/447; G01N 33/76

(52) U.S. Cl. CPC B01L 3/50273; G01N 27/026; G01N 27/44791; B01L 2400/0424; B01L 2300/0636; B01L 2300/0867; G01N 33/76

(57) ABSTRACT

An embodiment in accordance with the present invention is directed to a non-optical, label-free microfluidic biosensor utilizing an electrical liquid interface between two co-flowing liquids—one with a higher conductivity and one with a higher dielectric constant. The analyte-of-interest is in one solution while the receptor is in the adjacent stream. The electric interface acts as a substrate, when an alternating current electric field is applied perpendicularly across the interface, liquid displacement occurs which is frequency dependent. When a reaction occurs at the interface, it alters the electrical properties of the electrical interface, altering the frequency dependent liquid motion, which is then monitored by impedance spectroscopy downstream.

### CROSS REFERENCE TO RELATED APPLICATION

This application claims the benefit of U.S. Provisional Patent Application No. 62/307,200 filed on Mar. 11, 2016, which is incorporated by reference, herein, in its entirety.

### GOVERNMENT SUPPORT

This invention was made with government support under CBET-1351253 and CBET-1511185 awarded by the National Science Foundation. The government has certain rights in the invention.

### FIELD OF THE INVENTION

The present invention relates generally to diagnostics. More particularly, the present invention relates to a non-optical label-free biomolecular detection at electrically displaced liquid interfaces using interfacial electrokinetic transduction.

### BACKGROUND OF THE INVENTION

Biosensors combine targeted biological recognition with physicochemical transduction to detect specific biomolecules within a biological sample. They are used in a wide range of analytical applications, including diagnosis and treatment of infectious diseases, biowarfare detection, environmental monitoring, drug discovery, cell biology, cancer research, and point-of-care diagnostic testing. Here, a key challenge to developing label-free homogenous biosensors for sensitive biomolecular detection and kinetic analysis using microfluidic biosensing systems that normally require fluorescently labeled biomolecules and optical quantification is addressed.

Combining biosensors with microfluidic transducers can fulfill an increasing demand for fast, inexpensive sensors capable of molecular detection and analysis. Microfluidic biosensors provide several advantages over traditional laboratory-based detection methods, including faster analysis time, reduced sample and reagent consumption, and potential automation with sample processing units using on-chip microfluidic valves, pumps, mixers and detectors. In microfluidics, liquid transport usually occurs at low Reynolds number where the fluid flow is laminar; fluid streams flow side-by-side and mixing is driven only by diffusion.

Microfluidic liquid interfaces have been used as homogenous biosensing substrates for quantitative molecular detection and kinetic analysis in solution phase. The interface is created using laminar flow, where two streams combine at a fluidic junction and flow side-by-side down a main channel. One stream has a target probe and the second stream contains the biological sample of interest. Biorecognition occurs in solution phase as the target and sample streams diffusively mix at their contacting liquid interface where target and sample molecules specifically bind to one another. This approach offers an inexpensive, yet extremely powerful method for biosensing and biomolecular kinetic analysis, and has been used to quantify fast kinetic processes, extract kinetic rate constants, perform sensitive on-chip immunoassays and detect DNA hybridization reactions in solution.

To create a complete biosensor, the microfluidic interface is coupled with a transducing element to convert recognition events into a detectable measurement signal. Depending on the nature of the transduced biosensor signal, detection can be performed optically, electrically, or mechanically. Biosensing at microfluidic liquid interfaces, however, is currently only performed using optical methods such as fluorescent microscopy, fluorescence energy transfer (FRET), fluorescence correlation spectroscopy, confocal fluorescent microscopy, and fluorescence lifetime imaging microscopy (FLIM). While effective, they require fluorescently labeled probe molecules and optical components, which can significantly increase the cost and size of the microfluidic platform.

Microfluidics offers an attractive platform for performing miniaturized chemical and biomolecular analysis. Particularly useful is the ability to embed multiple laboratory steps, including preparation and chemical detection, into a microfluidic chip for automated sample processing and analysis. An important engineering design challenge in microfluidics is to develop new in situ analytical tools that monitor these operations within the confines of the microchannel network. Fluorescent-microscopy, for example, has been used to perform noninvasive quantification of liquid properties and flow velocity in micro channels. Other techniques such as in situ Ramen spectroscopy, imaging FTIR and cyclic voltammetry have been used to capture microscale chemical images of microfluidic channel surfaces and fluidic flow patterns. Another strategy relies on employing electrochemical impedance spectroscopy (EIS) to monitor electrical changes on microfluidic surfaces and cells in contact with electrolyte. Because EI Scan be performed with micro-electrodes integrated on-chip, it has potential to be a powerful tool for rapid non-optical monitoring of microfluidic processes. EIS is used for monitoring complex surface processes in microfluidic space without the use of fluorescent labels and fixatives. By detecting variations in impedance as a function of field frequency, EIS, for example, can be used to non-optically detect proteins and DNA through monitoring protein binding and DNA hybridization on electrode surfaces, where these interactions influence current flow and the corresponding impedance spectrum. EIS can also be used to quantify cell growth. Cells, for example can be grown on the surface of patterned EIS micro-electrodes. The electrode impedance has been shown to be influenced by the cell type and cell state. Because EIS can be easily integrated into microfluidic flow channels, its utility could be extended to non-optically monitoring fluidic behavior for monitoring on-chip mixing and routing operations where multiple fluid streams are routed, mixed, http://dx. and pumped across a network of micro channels.

Accordingly, it would be advantageous to provide a non-optical, label-free biomolecular detection at electrically displaced liquid interfaces using interfacial electrokinetic transduction.

### SUMMARY OF THE INVENTION

The foregoing needs are met, to a great extent, by the present invention which provides a non-optical, label-free microfluidic biosensor device for biomolecular detection of an analyte-of-interest including an electrical, liquid interface between two co-flowing liquids. The device also includes a source of alternating current applied to the electrical-liquid interface in order to produce frequency-dependent liquid motion, upon which the reaction at the interface alters the frequency response.

In accordance with an embodiment of the present invention, one of the two co-flowing liquids has a high conductivity relative to the other, and one of the two co-flowing liquids has a high dielectric constant relative to the other. The analyte-of-interest is included in a stream of one of the co-flowing liquids, while a receptor for the analyte-of-interest is included in the other stream. The alternating current is applied perpendicularly across the interface causing frequency dependent liquid displacement. A device for impedance spectroscopy is positioned downstream from the electrical-liquid interface for monitoring alteration in the frequency-dependent liquid motion.

In accordance with an embodiment of the present invention, a method for non-optical, label-free microfluidic, biomolecular detection of an analyte-of-interest in a sample includes generating an electrical, liquid interface between two co-flowing liquids. The method also includes applying alternating current to the electrical-liquid interface to produce frequency-dependent liquid motion. A reaction alters the frequency response.

In accordance with an aspect of the present invention, one of the two co-flowing liquids has a high conductivity relative to the other and one of the two co-flowing liquids has a high dielectric constant relative to the other. The analyte-of-interest is included in a stream of one of the co-flowing liquids and including a receptor for the analyte-of-interest in another stream. The alternating current is applied perpendicularly across the interface causing frequency dependent liquid displacement. The method includes monitoring electrical properties of the electrical-liquid interface. Monitoring of the electrical properties of the electrical-liquid interface is done using a device for impedance spectroscopy. The device for impedance spectroscopy is positioned downstream from the electrical-liquid interface. The method includes displaying a presence of the analyte-of-interest in the sample. The method can also include detecting female hormones.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIGS. 1A-1C illustrate image and schematic diagram views of an electrokinetic approach, according to an embodiment of the present invention.

FIGS. 2A-2D illustrate image views of fluorescent labeled fluids, according to an embodiment of the present invention.

FIGS. 3A-3D illustrate graphical and image views of frequency measurements associated with an embodiment of the present invention.

FIGS. 4A and 4B illustrate graphical and image views of interface displacement and position, according to an embodiment of the present invention.

FIG. 5 illustrates a graphical view of an IET sensorgram illustrating selective sensor response to both BSA/anti-BSA and avidin/biotin binding, according to an embodiment of the present invention.

FIGS. 6A-6D illustrate sensorgrams for avidin-biotin binding, according to an embodiment of the present invention.

FIG. 7 illustrates a flow system according to an embodiment of the present invention.

FIG. 8 illustrates an image view of a device according to an embodiment of the present invention. The image includes a dime for scale.

FIGS. 9A-9D illustrate image views of a confocal micrograph with two embedded electrode arrays.

FIGS. 10A-10C illustrate image views of top-down confocal micrographs of the interface position take above the impedance sensing electrodes for three different electric field frequencies applied at the upstream displacement electrodes.

FIG. 11 illustrates graphical views of the magnitude of the impedance |Z| versus applied excitation frequency for different fDEP interface positions.

FIG. 12 illustrates a graphical view of a magnitude of the impedance for different fDEP frequencies at an impedance excitation frequency of 500 kHz.

FIGS. 13A and 13B illustrate graphical views of impedance take ant an excitation frequency of 500 kHz versus applied fDEP frequency for two different interfacial conductivity differences.

FIGS. 14A-14C illustrate schematic and image views of the microfluidic T-channel.

FIGS. 15A-15F illustrate graphical views of fDEP crossover frequency.

FIGS. 16A-16D illustrate schematic and graphical views of an IET bead-based bioassay.

FIG. 17 illustrates a graphical view of Streptavidin Limited Detection by Varying Particle wt % and a sensorgram illustrating the decrease in signal with decreasing weight percent of streptavidin-coated particles.

FIG. 18 illustrates a graphical view of biotin detection in a BSA background and a sensorgram of the ability of the fDEP biosensor to detect biotin-streptavidin binding in a background of 5 mg/mL BSA.

FIG. 19 illustrates a graphical view of detection of human IgG protein A binding.

FIG. 20 illustrates a graphical view of influence of binding reaction on electrical properties measured by interface displacement.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention takes the form of a non-optical, label-free microfluidic biosensor utilizing an electrical liquid interface between two co-flowing liquids—one with a higher conductivity and one with a higher dielectric constant. The analyte-of-interest is in one solution while the receptor is in the adjacent stream. The electric interface acts as a substrate, when an alternating current electric field is applied perpendicularly across the interface, liquid displacement occurs which is frequency dependent. When a reaction occurs at the interface, it alters the electrical properties of the electrical interface, altering the frequency dependent liquid motion, which is then monitored by impedance spectroscopy downstream. Any other means of detection known to or conceivable to one of skill in the art could also be used.

Current state of the art detection methods rely on solid surface substrates, however this leads to several complications involving fouling, non-specific binding, single-use, complex surface chemistry as well as multiple washing and rinsing steps. Many of these methods are also limited to complex lab equipment increasing the cost while decreasing the ease-of-use and portability. The present invention creates a liquid-liquid interfacial substrate, eliminating many complications due to conventional surface substrates. Liquid-Liquid substrates are well known in microfluidics, yet they require labelling, which in turn requires the use of optics. Briefly, an antibody, ssDNA or more generally receptor for the analyte-of-interest has a fluorescent tag, which fluoresces upon binding. This leads to complex labelling techniques, as well as added costs. This invention is the first non-optical, label-free liquid-liquid interfacial biosensor. The present invention also allows for much faster detection at the same level of specificity. Indeed, the present invention can provide results at a rate that is two logarithm faster than previous detection methods, while providing the same specificity. The substrate is the liquid-liquid electrical interface. An AC electrical field polarizes the interface which leads to liquid displacement, this displacement becomes the transducer for the biosensor. Finally, there exists a frequency where no displacement occurs, known as the crossover frequency, which is dependent on the electrical properties of the liquid electrical interface. The change in crossover frequency due to biomolecular binding at the liquid interface becomes the signal. By integrating an impedance sensor downstream, the position of the liquid interface can be monitored. This position will change depending on the frequency applied. At a specific applied frequency, the magnitude and direction of displacement is dependent on electrical properties of the liquid interface, which change due to product formation.

The present invention is directed to a label-free homogeneous electrokinetic biosensor for detecting and quantifying bioaffinity interactions in solution. The approach combines continuous microfluidic flow with alternating current (AC) electrokinetics. Electrokinetics integrates well with microfluidics and is a useful tool for a variety of on-chip fluidic applications including cell manipulation, liquid mixing, particle trapping and fluidic routing. One feature of low Reynolds number (Re) flows is that multiple liquid streams can flow side-by-side without convectively mixing. The result is that well-controlled microfluidic laminar interfaces can be created between both miscible and immiscible liquids. Interfacial flows are ubiquitous in low Re systems and play important roles in microfluidic applications in rheology, chemical detection, molecular mass sensors, immunoassays, DNA hybridization, and kinetic analysis. The present invention can detect with the same sensitivity at a rate that is approximately two logarithm faster than prior detection methods. The present invention can be configured to detect any number of analytes-of-interest known to or conceivable to one of skill in the art, one exemplary category of analytes is female hormones.

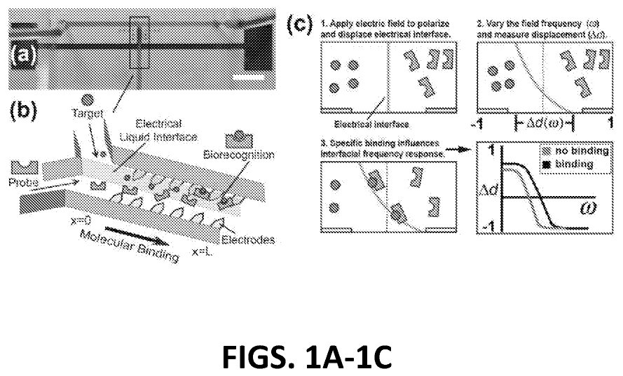

In the electrokinetic approach, depicted in FIGS. 1A-1C, bioaffinity binding occurs at a microfluidic liquid interface. The interface includes a microfluidic T-channel device with two fluid inlets and a single fluidic outlet; two fluids combine at the channel junction and create an interface as they flow side-by-side down the main channel to the outlet (FIG. 1A). An array of microelectrodes is fabricated in the main flow channel and used to deliver a polarizing electric field across the fluid interface. To be polarizable, and capable of being manipulated with the applied electric field, an electrical mismatch is engineered into the interface. In this way, the interface is an electrical interface (EI), comprised of two co-flowing fluid streams with different electrical conductivity (σ) and permittivity (ε). Using the EI as a substrate for biomolecular reaction, two solutions—one containing a target protein and the other containing an analyte probe—are pumped into separate T-channel inlets at a constant flow rate, inter-diffuse across the EI and specifically bind at the liquid interface (FIG. 1B). During this process, an AC electrical field is applied across the EI. Due to the large electrical mismatch, the interface polarizes and electrokinetically displaces across the main flow channel, perpendicular to the main flow direction (FIG. 1C). The magnitude and direction of this displacement is proportional to the electrical mismatch at the EI. Here, specific biomolecular interactions influence this mismatch, and allow for biomolecular interactions to be detected and quantified without fluorescent labels by measuring the interfacial displacement under an external electric field during binding (FIG. 1C). A device according to the present invention can include a display for showing the detection of the analyte of interest and the content of the analyte of interest in the sample. Such a display can be integrated into the device or can be displayed on an associated computer or other computing device, known to or conceivable to one of skill in the art. Results can be tracked and recorded for analysis and charting.

FIGS. 1A-1C illustrate image and schematic diagram views of an electrokinetic approach, according to an embodiment of the present invention. FIG. 1A illustrates an image view of a microfluidic T-channel device with integrated electrodes used to create an electrical liquid interface for the IET sensor. Scale bar 5.0 mm. FIG. 1B illustrates a schematic diagram of target-probe binding along the liquid interface; and FIG. 1C illustrates a schematic flow diagram of principles of IET sensor operation—(1) specific binding occurs at the electrical interface; (2) an electric field polarizes and displaces the interface a distance Δd, which is a function of the field frequency; (3) binding influences the polarizability of the interface and is detected by measuring interface frequency response at different values of field frequency.

The label-free biosensing method of the present invention uses the liquid interface as a homogenous substrate for specific binding, and its motion in an AC electric field as the transducer for biomolecular recognition. Therefore, the present invention is directed to a new class of biosensors based on interfacial electrokinetic transduction (IET): specific binding changes the electrical properties at the EI, which is electrokinetically transduced and detected by measuring perturbations in field-induced fluid displacement.

In an exemplary implementation included to illustrate the invention and not meant to be considered limiting, IET is used to monitor avidin-biotin binding kinetics in real-time without labels. Moreover, the IET biosensor is specific and sensitive, and able to detect as low as 250 femtomolar avidin concentrations against a 5 mg/mL background of bovine serum albumin (BSA). The present invention provides for a methodology is established for rapid label-free IET biosensing at electrical liquid interfaces, demonstrate sensor performance and sensitivity for the detection of biomolecules, and measure avidin-biotin binding kinetics with millisecond time resolution. The electrokinetic sensor of the present invention provides a low-cost, rapid, and portable biosensing system for label-free real-time kinetic analysis and on-site biomolecular diagnostics in free solution.

Experiments were performed using the microfluidic T-channel device shown in FIG. 1A. The microfluidic T-channel device was fabricated using standard soft photolithography and microfabrication techniques. First, microchannel electrodes were fabricated using wet chemical etching. Glass cover slips (50×30 mm², No. 1, Fisher Scientific) were coated with 2 nm of chromium and 50 nm of gold using electron beam evaporation. The cover slips were patterned with photoresist (Shipley 1813) and exposed metal was etched using gold and chromium etchant creating an array of patterned metal electrodes. The microchannel was fabricated in PDMS (Momentive, RTV 615A). A 10:1 mixture of PDMS elastomer and curing agent was poured atop the wafer and baked at 85° C. for 50 min. The PDMS was gently peeled off the wafer and fluid ports were punched with a 0.75 mm diameter biopsy punch (Ted Pella, Inc.). The PDMS microchannel and electrode pattern were then exposed to oxygen plasma (Jelight, Model 42A) and immediately aligned and sealed under an inverted microscope. As shown in FIG. 1A, the device includes a main flow channel 100 μM wide and 65 μM high. The embedded electrodes are axially separated by 20 μM and symmetrically bridge the channel width with a total length of 2.0 mm. Electrodes with sharp points are utilized in order to maximize the electric field strength across the liquid interface, as the sharp point serves to focus the electric field to the tip of the electrode.

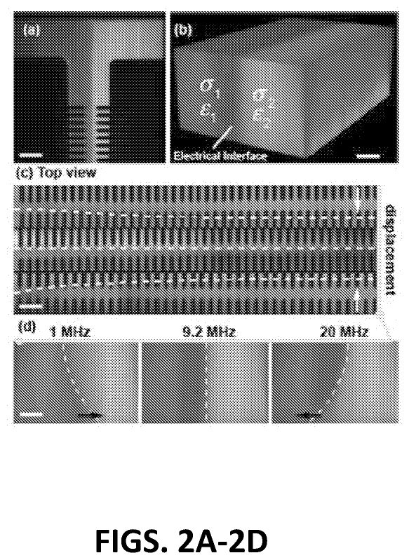

The electrical interface was created using the microfluidic “T-channel”. Two fluid streams were introduced into the device via pressure driven flow using an externally pressurized cryogenic vial. Shown in FIG. 2A, the left-most (dark grey) high conductivity stream is a 0.5 M phosphate buffered saline, pH 7.4, (PBS) solution with 10 ng/mL of Alexa Fluor 594 (Invitrogen). The right-most (light grey) high dielectric stream is a 0.8 M 6-aminohexanoic acid (Sigma-Aldrich) (AHA) labeled with 10 ng/mL of Alexa Fluor 647 (Invitrogen). AHA is a water-soluble zwitterion used for increasing the dielectric constant of aqueous solution. Prior to fluorescent labeling, the AHA solution is spun down in a centrifuge for 15 min in 1 g/mL Dowex MR-3 (Sigma) ion exchange resin to remove trace salts and reduce solution conductivity. A cross-sectional view of the resulting fluid interface was imaged using dual excitation confocal microscopy is illustrated in FIG. 2B.

FIGS. 2A-2D illustrate image views of fluorescent labeled fluids, according to an embodiment of the present invention. FIG. 2A illustrates a confocal micrograph of two fluorescently labeled fluids flowing side-by-side to create a liquid interface. Scale bar 50 μm. FIG. 2B illustrates a 3D confocal image stack of the electrical interface formed between to fluids with different electrical conductivities and dielectric constants. Scale bar 20 μm. FIG. 2C illustrates the interface enters the electrode array and displaces across the flow channel. The direction of this displacement is dependent on the frequency of the applied electric field. Scale bar 50 μm. FIG. 2D illustrates a confocal micrograph illustrates a side-view of the interface at different electric field frequencies. Scale bar 20 μm.

To create the electrical interface two fluid streams, each with a different set of electrical properties, are pressure injected into the microfluidic device. An AC potential of 10 V peak-to-peak (Vₚₚ) at a frequency of 1 MHz was dropped across the electrodes, and slowly is increased to 20 MHz while continuously monitoring the displaced position of the fluid interface.

Biotin, avidin, bovine serum albumin, and mouse anti-bovine serum albumin were purchased from Sigma Aldrich, USA, and used as received. A 16 μM biotin solution was made by diluting a 4 mM stock with AHA and labeled with 10 ng/ml Alexa Fluor 594, and pH adjusted to 7.4. The avidin solution was made by adding powdered avidin to PBS labeled with 10 ng/ml Alexa Fluor 647. The conductivity of the PBS solution was adjusted to 0.25 mS/cm using DI water. All subsequent solution avidin concentrations were made by serial dilutions with a stock dilute PBS. The final avidin concentration was calculated using a UV spectrometer (Thermo Scientific Genesys 10S).

IET biosensors are bioaffinity sensitized electrical liquid interfaces that electrokinetically displace in response to biomolecular binding. They require both microfluidic and electrokinetic components. The electrical interface is created using a PDMS microfluidic T-channel. An AC electric field is generated using an array of co-planar gold microelectrodes lithographically patterned onto the surface of a glass slide. The PDMS-electrode assembly is aligned under an optical microscope and plasma bonded to create a complete electro-fluidic device, as illustrated in FIG. 1A. The EI is composed of two co-flowing electrolyte streams. Both streams—streams 1 (left) and 2 (right)—have finite electrical conductivity and permittivity, but one has a greater electrical conductivity and the other a greater electrical permittivity such that σ1>σ2 and ε2>ε1. Because the streams do not mix except by diffusion, a sharp electrical mismatch is created at their co-flowing interface, which can be polarized (e.g. charged) and displaced across the microchannel using a perpendicular AC electric field.

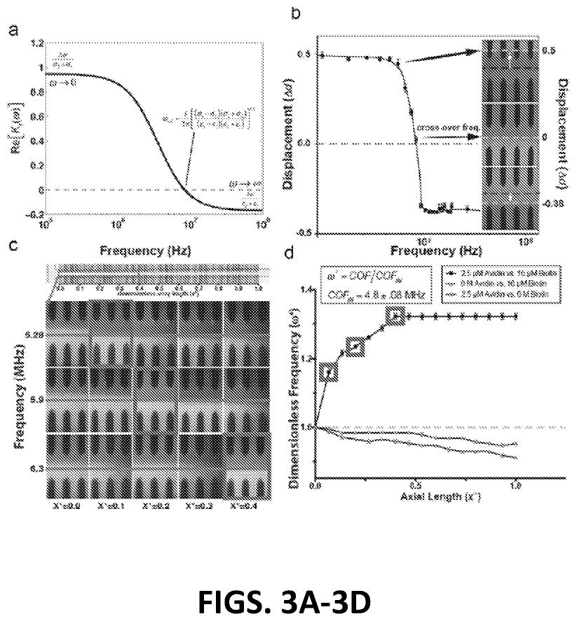

To quantify displacement, the interface position is imaged using confocal microscopy; each stream is labeled with a different fluorescent markers—Alexa Fluor 594 (dark grey) or 647 (light grey)—and imaged, yielding top-down 2D (FIG. 2A) and 3D (FIG. 2B) micrographs of the interface and its position within the microchannel. When a perpendicular AC electric field is applied, the interface displaces across the channel in a direction and magnitude dependent on the field frequency (ω). This displacement response can be confocal imaged and quantified from a top-down and side view. FIG. 2C shows a top-view of the EI displacement over the length of the electrode array for three different applied frequencies: ω=1 MHz, 9.2 MHz, and 20 MHz. At a frequency of 1 MHz (FIG. 2C-bottom), the interface enters the array and continuously displaces across the main flow channel into the high permittivity flow stream. At high frequency (20 MHz), the displacement direction reverses (FIG. 2C-top). At an intermediate crossover frequency (COF) of 9.2 MHz, the interface does not displace in the electric field and remains stationary over the entire length of the array (FIG. 2C-center).

The force driving interface motion is a surface force that exists over the separation length scale between the electrodes in the array. Because this length scale (20 μm) is smaller than the microchannel height (100 μm), field-driven displacement is localized to the bottom of the microchannel. Fluid near the top of the channel is driven in the opposite direction to satisfy conservation of mass, and the interface appears to tilt to the left or right depending on the applied field frequency. FIG. 2D shows the side-view of the displaced tilted interface for each applied field frequency.

The frequency response of the interface is used as a biosensing transducer for detecting biomolecules at a microfluidic liquid interface. To accomplish this, the net displacement of the EI is measured as a function of applied electric field frequency at the bottom surface of the microchannel. FIG. 3B shows the complete frequency response of the interface calculated from the micrograph experiment presented in FIGS. 2A-2D. The displacement (Δd) has been rendered non-dimensional over the microchannel width, and varies from −1 to 1. At the COF, the interface does not displace: Δd=0. Above and below the COF the displacement is finite and varies in both direction and magnitude with the applied frequency.

FIGS. 3A-3D illustrate graphical and image views of frequency measurements associated with an embodiment of the present invention. FIG. 3A illustrates a graphical view of liquid interface polarization[Eq. (1)] plotted as a function of field frequency. Low frequency polarization is driven by differences in electrical conductivity, while the interfacial dielectric constant governs the polarization of the interface at high frequency. A cross over frequency [Eq. (2)] is dependent on both interfacial conductivity and dielectric constant. The crossover frequency increases during biomolecular binding. FIG. 3B illustrates a graphical and image view of dimensionless interface displacement measured at different electric field frequencies. An optical micrograph highlights interface position at low (1 MHz), intermediate (9.2 MHz) and high (20 MHz) field frequencies; FIG. 3C illustrates optical micrographs of the electrical interface captured at different positions and electric field frequencies along the length of the electrode array. The interface crossover for each axial position is highlighted in blue and plotted in the adjacent figure; and FIG. 3D illustrates an IET sensorgram showing the influence of binding on interface crossover frequency plotted against two biosensor negative controls.

The motion of an electrical liquid interface in an externally applied AC electric field is known as fluidic dielectrophoresis (fDEP). Despite being discovered over six decades ago for particle suspensions, dielectrophoresis has only recently been applied to aqueous liquid interfaces. Here, the electrical and frequency dependence is defined on the interface displacement, and applied to the IET biosensor measurements.

For an EI subjected to a time varying monochromatic AC electric field, the magnitude and direction of the interfacial displacement is directly proportional to the interface polarization factor: K(ω). This factor describes the magnitude and sign of the field-induced ionic and dielectric charge that is induced at the EI in response to the electric field. Because the field oscillates monochromatically in time, the sign and magnitude of charge at the interface is dynamic, and reverses in phase with the electric field. This process takes a finite time, and depending on the field frequency, not all of the induced charge will be able to dynamically stay in phase. To account for this phase lag, K(ω) is a complex function with both real and imaginary parts, dependent on field frequency, and liquid conductivity and permittivity differences across the EI. Displacement is driven by the real part of this expression (i.e. interface charging that is in-phase with the applied field). The out of phase (imaginary) part produces a net interfacial electric stress with a zero time-average and does not contribute to interfacial motion. Therefore, the real part of the polarization factor is used with respect to the present invention.

For an electrical interface composed of two co-flowing aqueous electrolytes with different electrical conductivities and permittivities, the real part of the interfacial polarization factor is

$$Re [ K ( ω ) ] = \frac{( {e}_{2} - {ɛ}_{3} ) {r}^{2} {ω}^{2}}{( {e}_{2} + {ɛ}_{1} ) ( {s}^{2} {ω}^{2} + 1 )} + \frac{( {σ}_{2} - {σ}_{2} ) {r}^{2} {ω}^{2}}{( {σ}_{2} + {σ}_{1} ) ( {r}^{2} {ω}^{2} + 1 )} . \qquad ( 1 )$$

where

$$τ = \text{?} τ = ( ɛ 2 + ɛ 1 σ 2 + σ 1 )$$

$$\text{?} \text{indicates text missing or illegible when filed}$$

is the characteristic charge relaxation timescale at the interface between the two liquids.

Illustrated in Eq. 1, fDEP provides a unique method for quantifying the electrical properties of an EI because displacement direction and magnitude are both dependent on the relative electrical property mismatch between the interface's two co-flowing fluid streams. In FIG. 3A, Eq. 1 is plotted as a function of electric field frequency, and highlight the influence of electrical conductivity and permittivity mismatches at high, intermediate, and low AC field frequencies. At low frequency below the COF, polarization is driven by differences in electrical conductivity between the two co-flowing fluids (σ1-σ2), which is defined here as interfacial conductivity (Δσ). Above the COF at high frequency, displacement is governed solely by interfacial permittivity (Δε). The COF (ω_{COF}) occurs where the net polarization and displacement of the interface is zero (FIG. 3B). It is sensitive to both interfacial conductivity and permittivity, shown here by setting the polarization factor equal to zero (Re[K(ω)]=0), and solving for frequency:

$${ω}_{COF} = \frac{1}{2 π} [ \frac{( {σ}_{1} - {σ}_{2} ) ( {σ}_{3} + {σ}_{2} )}{( {ɛ}_{2} - {ɛ}_{1} ) ( {ɛ}_{2} + {ɛ}_{1} )} ] \text{?} \\ \text{?} \text{indicates text missing or illegible when filed} \qquad ( 2 )$$

Depicted in FIG. 3A, interface displacement at low and high frequency is influenced only by interfacial conductivity and permittivity, respectively, and the COF is a function of both properties. The transducing element of the IET biosensor is based on the interface COF. First, because the interface COF is influenced by both EI conductivity and permittivity, it is a single frequency measurement capable of monitoring both of these properties simultaneously at a liquid interface. Second, because the COF occurs when interfacial displacement is zero, it is a simple measurement to observe, and can be made at any axial position along the length of the electrode array.

To detect specific binding at a liquid interface, the EI is sensitized with target probe molecules and forced to flow adjacent to a sample stream containing an analyte. With developed and continuous flow down the T-channel, the fluid at each axial position within the main channel has a different average residence time, and the binding process will be at different time points of diffusive-reactive transport. To monitor binding dynamically during this process, the COF at the EI is quantified at discrete axial positions down the length of the main flow channel. Changes in EI electrical properties during binding influence the COF. Because specific binding progresses forward in time as fluid flows down the length of the array, biomolecular binding can be electrokinetically quantified in time by measuring the COF at varying positions in axial space down the channel length.

The IET biosensor signal was the COF of the liquid interface. This measurement is performed at discrete positions over the entire axial length of the microelectrode array to detect biomolecular binding dynamically in time. Biotin-avidin is used as the model system for studying the biosensor response. Avidin binds up to four molecules of biotin with high specificity and affinity, and is a useful binding model for characterizing biosensing systems. To more clearly compare sensor performance against different concentrations of avidin, both experimental variables—the COF and the array position—were rendered dimensionless. Shown in FIG. 3C, the electrode array was rendered dimensionless by its total length (2.0 mm); COF measurements were quantified along a dimensionless axial variable x*, spanning the domain {0, 1}. To maintain consistent reaction residence times within the electrode array, the fluid flow rate was fixed at 5.0 [symbol] . All biosensing experiments were performed using buffers with constant conductivity and permittivity. Because these properties were constant, the COF at x*=0 was fixed: COF_{IN}=4.8 MHz±1%. To track the evolution of the COF over the axial position of the array, the COF was rendered dimensionless (ω*) by COF_{IN} such that ω*=1 at x*=0.

To determine if specific avidin-biotin binding influences the COF of the interface, a COF sensorgram was captured using 2.5 μM avidin flowing adjacent to 16 μM biotin. Two negative controls were used for this experiment—one COF sensorgram was taken without biotin, and a second without avidin. Finally, the COF sensorgram was measured along the EI within the electrode array with both avidin and biotin. FIG. 3D shows the ω*vs. x* sensorgram from each experiment. In the absence of binding, the interfacial COF decreases by ˜8% over the microchannel length. During avidin-biotin binding, however, the interface COF increases by over 30%, reaching an equilibrium value of ω*=1.3 at a distance x*=0.4 down the array.

The polarized interface behaves as a biosensor transducer; specific binding influences the interfacial electrical properties, which are transduced electrokinetically as a change in COF for a given position down the electrode array. The COF changes dynamically, transducing biomolecular binding events over axial length as they proceed forward in time. This concept is reflected in the sensorgram data illustrated in FIG. 3D, where the COF appears as a binding curve, increasing over the array length and plateauing as the reaction saturates at the EI. The transduction properties of the EI can be observed optically, as depicted in the confocal micrographs shown in FIG. 3C. As the field frequency is increased, the axial position where no interface displacement occurs shifts. The COF at x*=0.1, for example, is 5.28 MHz; no displacement is observed at this point in the array. This frequency is above COF_{IN} (4.8 MHz) at x*=0 and below the COF for any position where x*>0.1, so the interface deflects in opposing directions surrounding this inflection point. As the applied frequency is increased, the position of the inflection point shifts, corresponding to a new position-dependent COF. These COF inflections are highlighted with boxes in FIG. 3C. They represent COF measurements for three axial positions within the electrode array, and correspond to the sensorgram data points emphasized with square boxes in FIG. 3D.

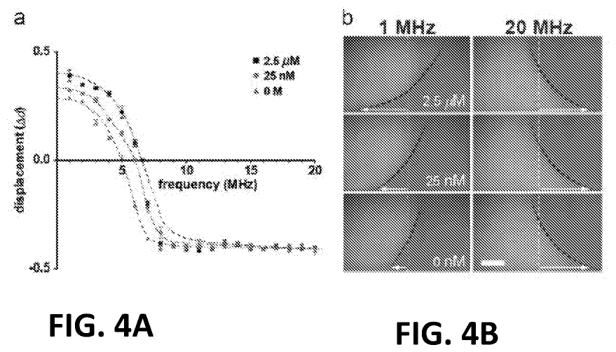

The response of the IET sensor is based on the influence of bioaffinity binding on the interfacial electrical properties at the EI. Because the COF is sensitive to both interfacial conductivity and permittivity, COF measurements are not enough to determine the exact electrical influence that biomolecular binding has on the interface. To determine how binding influences the electrical properties across the interface, the net displacement during binding over varying AC field frequency was measured at the saturation position x*=0.4 down the electrode array. FIG. 4A shows interfacial displacement spectrum as a function of field frequency for three different avidin concentrations: 0 nM, 25 nM, and 2.5 μM. The electrical mismatch of each of the fluids containing both avidin and biotin were held constant for each experiment. As shown above in FIG. 3A, low frequency displacement is dependent solely on the interfacial conductivity and depends only on interfacial permittivity at high frequency. From the spectra presented in FIG. 4A, avidin-biotin binding influences the low frequency displacement measurements—displacement increases with avidin concentration. At high frequency, however, interfacial displacement remains constant and is not influenced by interfacial binding. FIG. 4B shows a series of confocal micrographs depicting this observation. At 1 MHz the displacement increases as avidin concentration increases (FIG. 4B—Left), and high frequency (20 MHz) displacement remains unaffected with binding (FIG. 4B—Right).

FIGS. 4A and 4B illustrate graphical and image views of interface displacement and position, according to an embodiment of the present invention. FIG. 4A illustrates interface displacement measured over increasing concentrations of avidin: 0 M, 25 nM, and 2.5 μM. FIG. 4B illustrates confocal micrographs illustrate interface position at low (1 MHz) and high (20 MHz) for increasing avidin concentrations. Scale bar 20 μm.

The displacement measurements in FIGS. 4A and 4B demonstrate that the biomolecular binding of avidin-biotin increases the interfacial conductivity (Δd increases at low frequency with binding), but not alter the interfacial permittivity (Δd remains unaffected at high frequency). While fDEP can give insight as to how the interface is being influenced electrically, it cannot currently provide any mechanistic information about why this increase is occurring. The increase in interfacial conductivity may be due to counter-ion release during binding. One possibility is that a diffuse layer of spatially confined counter ions surrounds the charged proteins in solution. During binding, ions are released, which could potentially increase the electrical conductivity in the vicinity of the interface, and produce an increase in COF. Another possibility could be due to a change in electrophoretic mobility due to the negative charge of the bound biotin molecule, which would make the complex a more effective charge carrier at the interface. Currently, however, the precise physical mechanism for the interfacial conductivity increase remains unknown.

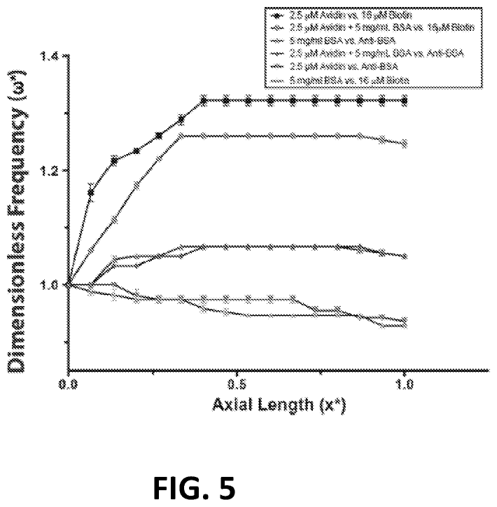

Surface-based heterogeneous biosensors can suffer from non-specific adsorption of background proteins, which can reduce sensor sensitivity. The IET sensor utilizes a liquid interface as a biorecognition substrate, and therefore is much less prone to suffer from biofouling or non-specific adsorption of proteins to the sensor surface. However, background interference from non-specific proteins is a major concern when working with real-world clinically relevant samples. To investigate sensor performance in the presence of an abundant background protein, the sensor was challenged with 5 mg/mL of bovine serum albumin (BSA). In order to be able to compare the findings with the avidin-biotin experiments performed without a background (FIG. 3D), the inlet COF and microchannel flow rate were held to less than a 1% deviation from their previous experimental values. Shown in FIG. 5, the COF increases about 25% over the length of the array when taken with 2.5 μM avidin flowing adjacent to 16 μM biotin in the presence of a serum background. To investigate the ability to design specific bioaffinity response into the EI, avidin was removed from the sample stream and replaced it with a 100 nM concentration of anti-BSA. Shown in FIG. 5, the sensor responds to the presence of BSA instead of avidin. Two control experiments were performed to ensure these measurements were specific—removal of anti-BSA and avidin from the sample stream does not produce an increase in the COF along the length of the array. FIG. 5 illustrates a graphical view of an IET sensorgram illustrating selective sensor response to both BSA/anti-BSA and avidin/biotin binding, according to an embodiment of the present invention.

There are several important features to note regarding the sensor performance depicted in FIG. 5. First, as shown, when compared to the sensorgram without BSA, the selectivity of the sensor towards avidin in the presence of serum decreases by ˜30%. This decrease could be due to several factors. There is a possibility that non-specific charge-charge interactions between BSA and the surrounding biomolecules in solution could be influencing the interfacial conductivity and total concentration of avidin available for binding. However, as shown in the BSA sensorgram in FIG. 5, the addition of background avidin to the BSA solution during binding with anti-BSA does not affect the magnitude of the sensorgram. This suggests that non-specific interactions between BSA and avidin are minimal.

Because the sensor COF is driven by a combination of both target-receptor binding and interfacial smoothening by ionic diffusion, the addition of BSA hinders the rate of avidin diffusion towards and across the interface, which slows the rate of the binding and leads to a lower sensor COF. Finally, it is worth noting that the reaction between BSA and anti-BSA does not produce the same change in COF when compared to the avidin and biotin reaction. This difference is due to the difference in binding kinetics between these two reactions. In comparing the K_{d} of each reaction, 10⁻¹⁵ and 10⁻⁴, for the avidin-biotin and BSA—anti-BSA reaction, respectively, the reaction between BSA and anti-BSA occurs at a slower rate than that of avidin and biotin. Since the reaction time is slower, binding requires a longer distance down the axial length of the microchannel. Because the diffusion of ions across the electrical interface is constantly occurring and decreasing the interfacial conductivity, the reaction requiring a greater length scale must compete with an ever-decreasing interfacial conductivity, which ultimately leads to a smaller magnitude in the change of COF. Future work will focus on developing a better understanding of these physicochemical mechanisms that link species reaction and diffusional rates with interfacial conductivity in order to better optimize sensor response.

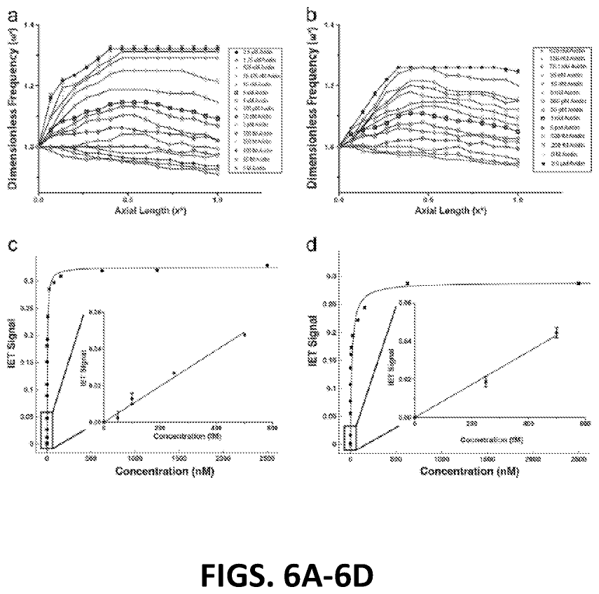

A series of experiments were performed to determine the IET sensor's limit of detection (LOD) and how the IE performs as a transducer against an abundant background protein (5 mg/mL BSA). The IET biosensor response was measured as a function of avidin concentration, ranging from 50 fM to 2.5 μM, both with and without BSA. FIGS. 6A-6D illustrate a series of sensorgrams for avidin-biotin binding at the EI, in FIG. 6A and with a BSA background in FIG. 6B. The net IET sensor response increases with avidin concentration, and eventually levels to a constant value some distance down the electrode array. The eventual leveling of the IET signal at a given position down the array length can be attributed to the saturation of the interface with bound avidin-biotin complex; avidin is depleted from the local EI and the sensor response saturates. FIGS. 6A-6D illustrate sensorgrams for avidin-biotin binding, according to an embodiment of the present invention. FIG. 6A illustrates an IET sensorgram of increasing concentrations of avidin. FIG. 6B illustrates a sensorgram of sensor response against a background of serum albumin. FIG. 6C illustrates a sensor binding curve showing IET limit of detection without a BSA background; and FIG. 6D illustrates a graphical view with a BSA background.

The magnitude of each saturated sensorgram response at a fixed distance down the electrode array (x*=0.5) was isolated and plotted as a function of avidin concentration. The resulting calibration curve is shown in FIG. 6C for an EI without BSA and in FIG. 6D for the interface subjected to a serum background. For each analytical curve, the sensor exhibits a linear response with increasing concentrations of avidin up to 500 fM, as shown in the subplots of each of FIG. 6C and FIG. 6D. Beyond this linear concentration range, the sensor deviates and begins to level off, eventually saturating at an avidin concentration at approximately 1 μM. The concentration LOD was calculated using the 3-sigma method (S/N=3) for each binding curve. Without background BSA, the sensor LOD was 209 fM, while the addition of BSA decreased sensor sensitivity with a corresponding LOD of 626 fM.

The present invention is directed to a sensitive and selective label-free electrokinetic biosensor for detecting biomolecules at electrically polarizable liquid interfaces. Biomolecular binding occurs at the diffuse electrical interface formed between two co-flowing microfluidic laminar streams with different electrical properties. The biosensor approach is based on measuring the electrical field-induced displacement frequency response of this interface, which is sensitively influenced by specific biomolecular binding. In this manner, the biosensor design utilizes this interface as substrate for biomolecular binding, and its motion in an electric field as a signal transducer. Binding increases the electrical conductivity at the interface, which is transduced as a change in interfacial frequency response, and forms the basis for the presented interfacial electrokinetic transduction (IET) method. The system developed can detect low femtomolar avidin concentrations against a 5 mg/mL background of serum albumin, and can be reconfigured to detect other proteins. The IET sensor has the potential be extended to other biomolecular systems for the detection of disease biomarkers in serum and urine. Furthermore, because binding occurs dynamically in time over the length of the microchannel interface, it should be possible to use this IET approach to study the binding kinetics of a variety of specific ligand-receptor pairs. Finally, while fluorescent microscopy was used in this work to measure interface position, future work will focus on developing inexpensive methods for measuring interface displacement electrically, extending the IET approach to more complex samples such as whole blood and urine, reducing interfacial diffusion of the electric interface, and developing reactive transport models to study binding kinetics at the liquid interface.

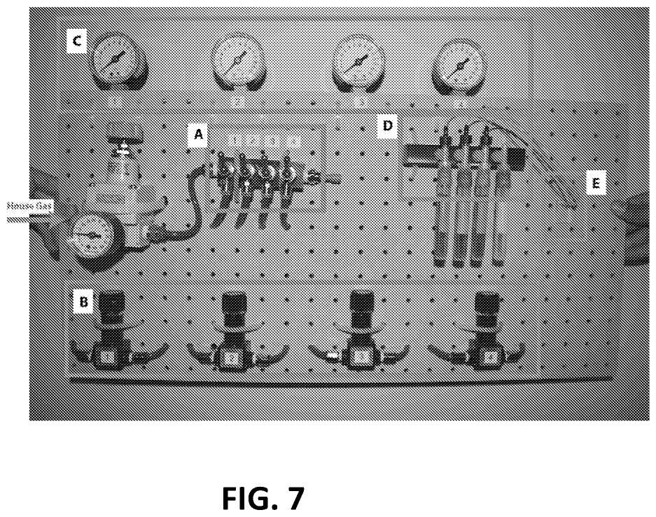

FIG. 7 illustrates a flow system according to an embodiment of the present invention. Briefly, house gas flows into a precision regulator and the pressure is controlled to a usable pressure (˜5-15 psi depending on application). The air then flows to a switching manifold (boxed and labeled ‘A’). The manifold is equipped with four 3-way switches, yet depending on the needs a manifold with more or fewer switches can be used. Each switch is labeled with a number from 1-4, this corresponds to all the numbered parts in each box, so every component numbered ‘1’ is connected, numbered ‘2’ is connected and so on. Looking again at the switching manifold (boxed and labeled ‘A’), if one of the switches is turned ‘on’ then the house gas will travel from the switching manifold to a second regulator (boxed and labeled ‘B’). These regulators offer more precise and tunable pressures at low values (1-30 psi). The gas passes from the regulator and is measured by a pressure gauge (boxed and labeled ‘C’). From the pressure gauges, the air travels to the sample manifold (boxed and labeled ‘D’).

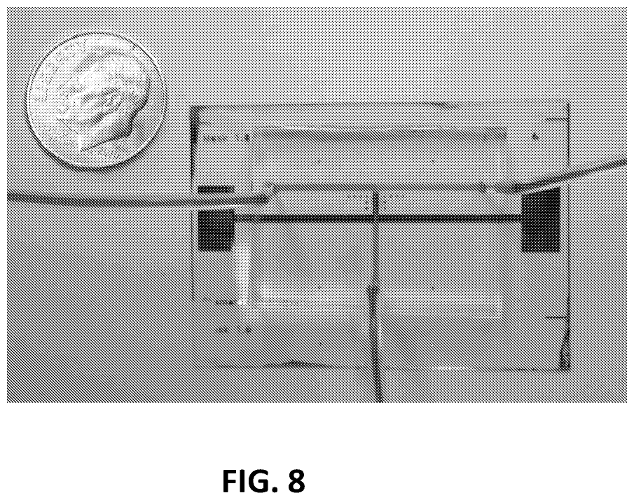

The sample manifold holds four cryovials, but again this number can increase or decrease depending on the needs. The house gas which has been precisely regulated now reaches the sample manifold and pressurizes its respective cryovial. From left to right the cryovials correspond to the parts numbered 1-4 respectively. There is tubing connected through the top of the cryovial, as pressure is applied in the cryotube the liquid has only one exit, through the tubing. The liquid travels through the tubing which is connected to the microfluidic device (boxed and labeled ‘E’). The flowrate of the sample is proportional to the applied pressure i.e. increasing the applied pressure increases the sample flowrate. FIG. 8 illustrates an image view of a device according to an embodiment of the present invention. The image includes a dime for scale.

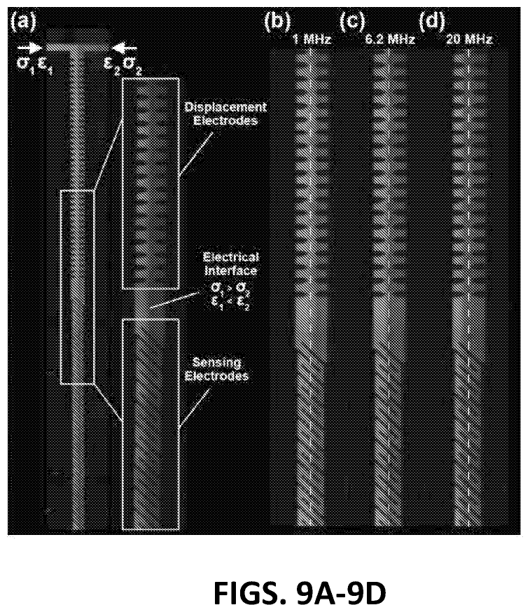

In another exemplary embodiment included to illustrate the present invention, but not meant to be considered limiting, the interface is created using a microfluidic T-channel device where two fluids are forced to flow side-by-side. Each liquid has a different electrical conductivity (σ) and dielectric constant (ε) such that a large electrical mismatch exists at their interface. fDEP motion is created using a perpendicular electric field produced from an array of parallel point electrodes integrated on the surface of the microfluidic channel (FIG. 9A). For a liquid interface subjected to a time varying monochromatic electric field, the displacement is directly proportional to the real part of the interface polarizability factor, K(ω), which is function of field frequency (ω), electrical conductivity and permittivity, as described above in Eq. (1). (1). As depicted in Eq. (1), because the interfacial polarization (e.g. charging) is driven by both conductive and dielectric charging, displacement is a function of the electrical properties of each fluid stream and the AC electric field frequency. At frequencies on the order of 100 kHz, for example, the magnitude of interface displacement is governed solely by differences in the electrical conductivity between the two co-flowing fluids, which is defined here as the interfacial conductivity (σ₂-σ₁). At high frequency (typically >10 MHz), however, the displacement is driven by the interfacial permittivity (ε₂-ε₁). Finally, at intermediate frequency the interface behavior is sensitive to both electrical and dielectric differences. Because of the low and high frequency attributes of fDEP, if one fluid phase has a greater conductivity (σ₂>σ₁) and the adjacent fluid has a greater dielectric constant (ε₂>ε₁), the direction of the interface displacement will reverse at a critical frequency and there will be a characteristic cross-over frequency (COF) where no interface displacement is observed. This occurs when the inter-face's polarizability factor is zero, which can be expressed as Eq. (2). At low frequency below the COF, the high conductive fluid displaces across the flow channel (FIG. 9B). At the COF the net charge on the interface is zero and no displacement is observed (FIG. 9C). Finally, above the COF the interface displaces in the opposite direction (FIG. 9D) and the low conductive high dielectric fluid displaces across the channel. FIGS. 9A-9D illustrate image views of a confocal micrograph with two embedded electrode arrays. FIG. 9A illustrates two co-flowing fluids with varying electrical properties are driven into a microfluidic t-channel device. Each inlet channel (75 μm in width) merges with a main 150 μm wide main channel to create a sharp liquid interface. One stream has a larger conductivity (light grey), while the adjacent stream (dark grey) has a large permittivity. The main channel has two embedded electrode arrays—displacement electrodes actuate the liquid electrical interface and sensing electrodes measures the local impedance. FIG. 9B illustrates a 1 MHz AC electric field applied to displacement electrodes, displacing the high conductive stream (light grey) into the low conductive stream (dark grey) A dotted white line shows the original interface position when no electric field is applied. FIG. 9C illustrates that at a COF of 6.2 MHz the interface does not deflect in the field. FIG. 9D illustrates that at 20 MHz the high dielectric stream (dark grey) displaces into the low dielectric stream (light grey).

The design of the exemplary embodiment requires a laminar interface and two different types of electrode arrays to displace and subsequently detect the interface position. A microfluidic “T-channel” device is used to create the liquid interface. Two fluid streams were supplied to the microfluidic device using a low-cost constant pressure source flow system. The microfluidic device was fabricated using standard soft photolithography and microfabrication techniques. Microchannel electrodes were fabricated using wet chemical etching. Glass cover slips (50×30 mm, no. 1, Fisher Scientific) were coated with 2 nm of chromium and 50 nm of gold using electron beam evaporation. The cover slips were patterned with photoresist (Shipley 1813) and exposed metal was etched using gold and chromium etchant. The resulting electrode pattern was then aligned and bonded to a soft lithographically fabricated T-channel device. To fabricate this device, the “T-channel” pattern was lithographically fabricated to a silica wafer using SU-8 3050photoresist (Microchem Corp.). A 10:1 mixture of polydimethyl-siloxane (PDMS) elastomer and curing agent was poured atop the wafer and baked at 85.0 for 30 min. The PDMS was gently peeled off the wafer and cut out of the mold. Fluid ports were punched with a 0.75 mm diameter biopsy punch (Ted Pella, Inc.). The electrode patterned coverslip was then exposed to oxygen plasma (Jelight, Model 42A), the PDMS microchannel was exposed using a handheld tesla coil (Electro-Technic Products Inc. Model BD-20) and the two substrates were immediately aligned and sealed under an inverted microscope. The assembled device includes a main flow channel 150 μm in width and 65 μm in height with an upstream displacement (parallel-point) and a downstream impedance (45°-interdigitated) electrode array (FIGS. 9A-9D). To perform an experiment, the fluid interface was subjected to an electric field using the parallel-point electrode array and forced to displace by fDEP at different field frequencies (FIGS. 9B-9D). As fluid exited the first displacement array, the interfacial stress ceased. Because the inertial influence on the flow is minimal (Re<1), the fluid interface remained fixed in a displaced position immediately after exiting the fDEP array. The deflection position is then determined by measuring the magnitude of the impedance using a second array of interdigitated electrodes.

The liquid interface was composed of two fluids, each with a different electrical conductivity (σ) and dielectric constant (ε). When forced to flow side-by-side at low Reynolds number these two fluids formed an interface with a large electrical mismatch between them. Each stream was injected at a constant flow rate (10 μL/min) into the device using a low-cost flow controller equipped with an externally pressurized fluid-filled cryogenic vial. Each fluid was labelled with a different Alexa Fluor fluorescent dye to accurately image the interface position using confocal microscopy.

Shown in FIGS. 9A-9D, the electrical interface was formed by flowing a left-most (light grey) 1×PBS solution (σ₁=0.29 mS/cm; ε₁=78) with 10 ng/mL of Alexa Fluor 488 (Invitrogen). The right-most (dark grey) high dielectric stream (σ₂=19 μS/cm; ε₂=110) was comprised of 0.8 M 6-aminohexanoic acid (Sigma-Aldrich) (AHA) labelled with 10 ng/mL of Alexa Fluor 594 (Invitrogen). AHA is a water-soluble zwitterion used for increasing the dielectric constant of aqueous solution. Prior to fluorescent labeling, the AHA solution was polished with 1 g/mL Dowex MR-3 (Sigma) ion exchange resin to remove trace salts and reduce solution conductivity. The COF of this electrolyte system was measured using previously published methods and found to be 6.2 MHz.

The upstream parallel-point array was used to drive fDEP flow across the channel and a second downstream 45°-interdigitated array as an impedance sensor. The parallel-point electrodes were axially separated by 20 μm and symmetrically bridged the width of the microchannel. Electrodes with sharp points are used to focus the electric field to the tip of the electrodes and to provide increased contact with the PDMS and glass substrate along the main flow channel walls. A function generator (Rigol DG4102) was connected to the fDEP electrodes and delivered an AC electric field to displace the interface across the channel. The downstream impedance electrodes were interdigitated and positioned at a 45° angle relative to the flow direction to maximize the sensitivity of the array to changes in interfacial position. An impedance spectrometer (Sciospec ISX-5) was connected to the impedance electrode array and used to measure the magnitude of the impedance as a function of interface position. For all impedance measurements, a sine-modulated AC potential of 50 mV was applied to the electrode array and the magnitude and phase angle of impedance were measured over an excitation frequency range between 100 kHz and 10 MHz. FIGS. 9B-9D depict a top-view of the interfacial motion of the interface over the length of the fDEP electrode array when a 10-Vpeak-to-peak (Vₚₚ) potential was applied across the displacement electrodes at three different field frequencies (1 MHz, 6.2 MHz, and 20 MHz). When the electric field frequency was 1 MHz, the conductive PBS (light grey) stream displaced across the interface (FIG. 9B). When the COF (6.2 MHz) was applied, both conductive and dielectric forces were equal, and the interface remained fixed as it passed through the electrode array (FIG. 9C). Finally, at a frequency above the COF (20 MHz) the deflection reversed direction and the high dielectric stream (dark grey) displaced across the microchannel (FIG. 9D).

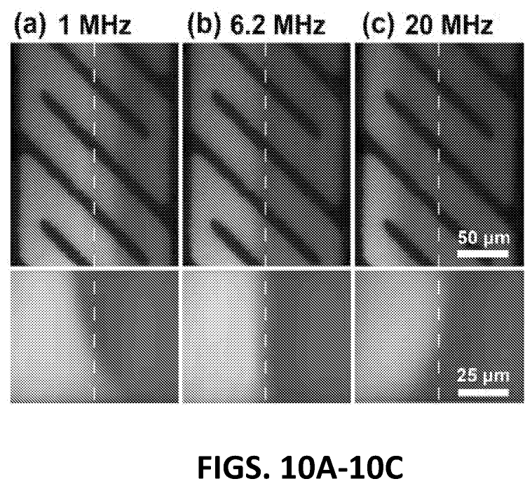

FIGS. 10A-10C illustrate image views of top-down confocal micrographs of the interface position take above the impedance sensing electrodes for three different electric field frequencies applied at the upstream displacement electrodes. A magnified 3D confocal z-stack is depicted below each top-down micrograph. For each image pair, a dotted white line highlights the interface position when no field is applied. FIG. 10A illustrate that at a field frequency of 1 MHz the high conductive stream (light grey) is driven across the channel surface. The corresponding 3D confocal stack shows an increase in conductive light grey fluid covering the surface of the sensing electrodes. FIG. 10B illustrates that the COF, 6.2 MHz, is applied and the liquid interface remains fixed. FIG. 10C illustrates that the direction of displacement reverses at 20 MHz and the high dielectric stream displaces into the low dielectric stream. The 3D image shows the direction reversed, leading to a decrease in conductive stream covering the impedance electrodes. Because the interfacial-driven motion of the fluid ceases upon exiting the displacement electrode array, the interface's displaced position can be accurately determined using the downstream impedance electrode array (FIGS. 10A-10C).

The top-view micrographs shown in FIGS. 10A-10C illustrate that when the interface was subjected to either a low or a high frequency electric field, a greater degree of either high conductive-low dielectric PBS or low conductive-high dielectric AHA covered the sensing electrodes at low and high field frequency, respectively. To observe the 3D structure of the interfacial flow field, 2D confocal micrographs were captured above the impedance electrode array for three different electric field frequencies: 1, 6.2 and 20 MHz (FIGS. 10A-10C). Because the displacement electrodes are thin co-planar films (˜52 nm) and confined to the microchannel surface, the electrical stress responsible for driving flow was localized near the surface of the microchannel. In order to satisfy mass conservation, this local electrokinetic flow was countered by a pressure driven back flow at the top of the channel which produced a “tilted” interface, as shown in the 2D confocal micrographs in FIGS. 10A-10C. Because the impedance electrode array is also confined to the microchannel surface, the impedance measurements were only sensitive to the electrical properties of the fluid domain very near the surface where the electric field was capable of penetrating into the liquid domain. Therefore, differences in interfacial position produced by fDEP will create changes in both the local conductivity and dielectric constant of the fluid near the impedance sensor.

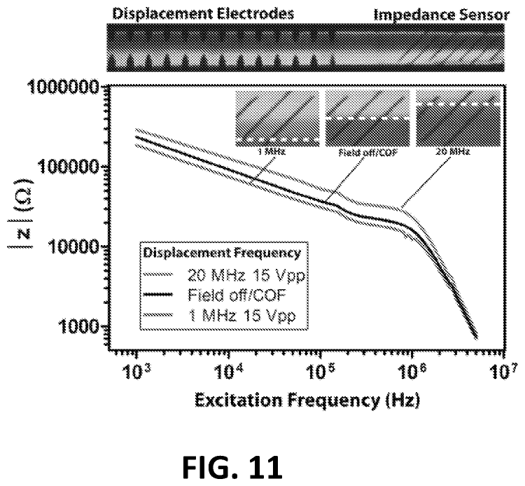

In order to determine the optimum impedance conditions for measuring interfacial position, upstream fDEP displacement influence on downstream impedance over a range of impedance excitation frequencies is determined. An electrical interface was created by co-flowing solutions of PBS and AHA, and then deflected at frequencies below (1 MHz) and above (20 MHz) the COF, and when no field was applied (e.g. the position at the COF). For each interface position, an impedance frequency sweep from 100 kHz to 5 MHz is performed to determine the magnitude of impedance for different interfacial positions (FIG. 11). FIG. 11 illustrates graphical views of the magnitude of the impedance |Z| versus applied excitation frequency for different fDEP interface positions. Three frequency sweeps were performed in order to determine the optimal frequency for the impedance spectrometer. When 1 MHz is applied to the displacement electrodes, the high conductive-low dielectric (green) stream covers more impedance sensor electrode area and the impedance magnitude decreases. When a 20 MHz electric field is applied the high dielectric-low conductive (dark grey) stream occupies a larger sensing electrode area and the impedance increases. The magnitude of impedance remains the same when the field is off and the COF is applied. Below the interfacial COF t 1 MHz, the fDEP electrodes polarized and forced the high conductive (light grey) stream to cover a larger area on the impedance electrode array. Conversely, when a high frequency is applied above the COF, the high dielectric stream displaced across the inter-face and the impedance sensor was exposed to fluid with lower electrical conductivity. The impedance data was consistent with the electrical changes that the interface position produces in the vicinity of the impedance electrodes. When high conductive-low dielectric PBS covered a greater portion of the impedance electrode array, the impedance decreased, while the opposite was seen when the low conductive-high dielectric AHA stream displaced across the impedance electrode array. Because the interface position is not influenced by the electric field at the COF, the magnitude of the impedance at the COF is identical the case when no displacement field was applied since the interface does not displace at the COF. Shown in FIG. 11, the interface deflection produced the greatest change in the magnitude of the impedance (|Z|) at an impedance frequency of ˜500 kHz. Based on these experiments, |Z| is measured at a frequency of 500 kHz for all subsequent experiments in this work.

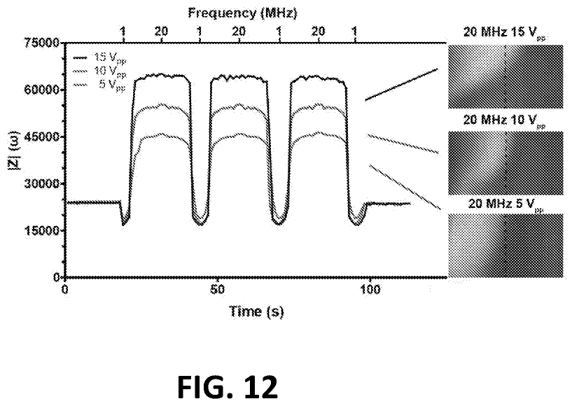

With the impedance excitation frequency fixed at 500 kHz, |Z| is next measured as a function interface position for three different applied voltages (5 Vₚₚ, 10 Vₚₚ, and 15 Vₚₚ) delivered across the interface using the upstream displacement electrode array. For each applied voltage the fDEP frequency was continuously swept from 1 to 20 MHz, and then back to 1 MHz while simultaneously measuring |Z| at the downstream impedance array. Shown in FIG. 12, when the interface is centered at the COF, |Z| was found to be 32.5 kΩ for all three voltages applied. FIG. 12 illustrates a graphical view of a magnitude of the impedance for different fDEP frequencies at an impedance excitation frequency of 500 kHz. However, when an fDEP frequency below the COF was applied, the high conductive PBS stream covered a larger portion of the impedance sensor surface and the impedance decreased from 25 kΩ to 15 kΩ at an applied voltage of 5 Vₚₚ. When the fDEP frequency increased above the COF, the high dielectric fluid covered a greater portion of the sensor surface and |Z| increased to 45 kΩ. The impedance was also influenced when the electrical interface was subjected to larger displacement voltages. This increase was particularly noticeable at high frequencies (>COF) where more low conductive, high dielectric AHA buffer covered a greater area of the impedance sensor. In order to visualize the interface, 3D confocal micrographs of the interface were captured when a20 MHz AC electric field was applied at voltages of 5, 10 and 15 Vₚₚ. Shown in the micrographs in FIG. 12, the AHA stream (red) covers a greater electrode area with increasing applied voltage. While there is a large change in impedance with increasing voltage at high frequencies, the three |Z| datasets are not as strongly influenced at low frequencies (<COF). This is because changes in the position of the high conductivity buffer on the impedance electrodes do not influence the impedance to the same magnitude as the AHA buffer. Lastly, when the interface COF was applied to the interface, the interface did not displace in the electric field, and therefore the impedance did not change with applied voltage.

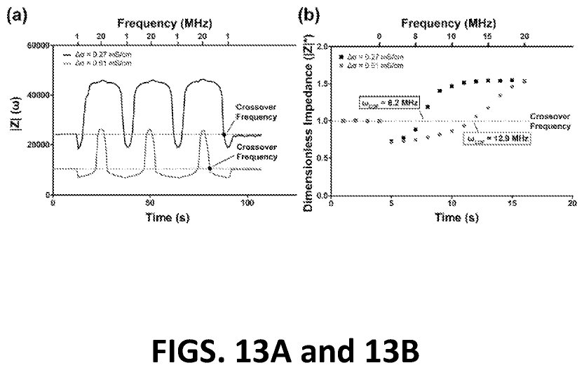

Next, it was determined if it was possible to distinguish the COF between two fluid interfaces with different electrical conductivity mismatches. In FIGS. 13A and 13B, impedance results are shown for two different interfacial conductivities—one where Δσ=σ₂−σ₁=0.27 mS/cm and a second interface where Δσ=0.61 mS/cm. Shown in FIGS. 13A and 13B a difference in measured impedance is observed for the two different interfaces, where the impedance measurements for a given interface position increased with interfacial conductivity. FIGS. 13A and 13B illustrate graphical views of impedance take ant an excitation frequency of 500 kHz versus applied fDEP frequency for two different interfacial conductivity differences. FIG. 13A illustrates a magnitude of impedance for varying applied frequencies 1-20 MHz. The interface with a lesser interfacial conductivity has a larger measured impedance. FIG. 13B illustrates a magnitude of impedance for both systems is rendered dimensionless to compare the two datasets. The impedance measurement is able to non-optically determine that the COF increases with increasing interfacial conductivity. For an alternative way to analyze these results, the impedance of each dataset is normalized by the initial impedance value when no electric field was applied using a time-averaged baseline impedance reading that was taken prior to applying an electric field at the displacement electrodes. Shown in FIG. 13B, both fluidic systems begin and end at the same dimensionless impedance, |Z|*, but their COF's are different and increase with interfacial conductivity, as is consistent with previous fDEP experiments.

Using an AC electric field the exemplary embodiment of the present invention, forced a laminar fluid interface to deflect across a microchannel using fDEP. The position of the deflected interface is then measured using a downstream impedance electrode array. The interface was composed of two co-flowing fluids—one stream had a greater electrical conductivity and the other had a greater dielectric constant. When the interface was exposed to a low frequency AC electric field, high conductive fluid stream displaced across the channel and covered a larger surface area of the impedance sensor, which reduced the magnitude of the impedance. As the frequency was increased to the COF, the interfacial position moved to its original position which reduced the amount of conductive fluid over the impedance array. At higher electric field frequencies above this COF, the interfacial deflection reversed direction and the high dielectric, low conductive stream covered a larger surface of the impedance sensor and produced an increase in impedance. This method was able to electrically detect interfacial displacement and measure the interface COF non-optically at a resolution consistent with experiments per-formed with confocal microscopy. This method provides a new sensing technique for non-optically imaging microfluidic flow fields.

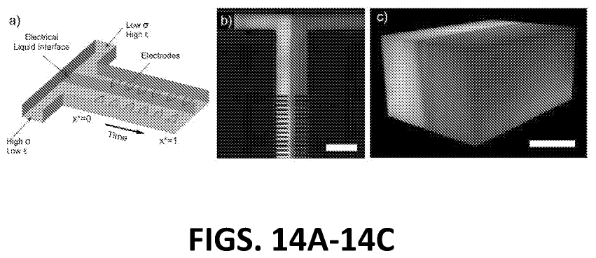

In another exemplary embodiment included to illustrate the present invention, but not meant to be considered limiting, the experimental device includes a combination of microfluidic flow, electrokinetics, and colloid-based biomolecular surface chemistry. FIGS. 14A-14C illustrate schematic and image views of the microfluidic T-channel. FIG. 14A illustrates the microfluidic T-channel with integrated electrodes. FIG. 14B illustrates a top-down confocal micrograph of the T-channel device with two-co-flowing fluorescently labelled fluids. Scale Bar 100 μm. FIG. 14C illustrates a 3D confocal z-stack of the microfluidic-generated electrical liquid interface. Scale bar 50 μm.

The device includes a liquid interface for binding by co-flowing two different fluid streams through a microfluidic T-channel. One stream contained a suspension of functionalized nanoparticles at a known weight per-cent (wt %), while the other contained target analyte molecules. To deliver an electric field across the interface, an array of parallel point microelectrodes was integrated within the main flow channel (FIG. 14A). The complete device, illustrated in FIG. 14B, was fabricated using a combination of microfluidic soft lithography and wet etching techniques. To fabricate the microarray of electrodes, glass cover slides (50×30 mm, no. 1, Fisher Scientific) were coated with a 20 nm layer of chromium, followed by a 30 nm layer of gold, using electron beam deposition. Positive photoresist (S1813, Shipley) was spun onto the gold-coated slides and exposed using a UV contact aligner with a positive mask (Fineline Imaging) before etching the gold and chrome layers with chemical etchants (Alfa Aesar). The electrodes were aligned symmetrically across the main flow channel axis, and spaced at 20 μm intervals, with pointed tips to focus the electric field at the liquid interface (FIG. 14B).

To fabricate the microchannel, a T-channel master mold was created by spin-coating negative photoresist (SU-8 3050, Microchem Corp.) on a 4-inch silicon wafer (Silicon Inc.) and exposed to a UV light source through a negative photomask. The microfluidic channels were created by pouring PDMS at a 10:1 elastomer to curing agent ratio over the wafer mold. The mold was baked at 85.0 for 30 min and the cured polymer was peeled off and cut to size. Finally, fluid ports were created with a 0.75 mm biopsy punch (Ted Pella, Inc.), and the PDMS was bonded to the glass slide by exposure to oxygen plasma (Mode142A, Jetlight) for 1 min and immediately aligned and sealed by eye under an inverted microscope. The completed device includes two fluidic inlet channels connected to a main microfluidic channel measuring 100 μm across and 35 μm high (FIG. 14B). Fluid flow was driven into the device using an external pressure source at a constant flow rate of 5 To create the electrical properties for interface deflection, it is necessary that one fluid stream have a greater dielectric constant than the neighboring fluid phase. For the binding experiments in this work, one fluid phase was com-posed of a 10× diluted phosphate buffered saline (PBS) solution and the adjacent phase was a 0.8 M solution of 6-aminohexanoic acid (AHA) (Sigma Aldrich). AHA is a water-soluble zwitterion intended to increase the dielectric constant of the aqueous solution.

To create a biosensing system at the liquid interface, a biomolecular recognition event was integrated between the two fluids, where the PBS stream contained a suspension of functionalized nanoparticles and the adjacent phase containing either the target analyte or a negative control. Both biotin and human IgG, and streptavidin and Protein A-functionalized nanoparticles were used as biomolecular systems to test the particle-based method. While these biomolecular systems are disclosed herein, they are not meant to be considered limiting and any particles known to or conceivable to one of skill in the art could be tested. Because of the strong binding affinity, biotin (Sigma Aldrich) and 100 nm streptavidin-coated silica nanospheres (ζ=−34.6 mV) (Corpuscular Inc.) were first used as a model bioreaction and 100 nm carboxylated silica nanospheres (ζ=−34.3 mV) (Corpuscular Inc.) were used as a non-reactive negative control. A 4 mM biotin stock solution was made in 0.8 M AHA and subsequently diluted to experimental concentrations ranging between 500 aM and 16 μM. The PBS stream contained the nanoparticle substrate: streptavidin-coated particles were triple washed and re-suspended in PBS solution and particle suspensions were maintained at 0.0375 wt % for all experiments unless specified otherwise. Human immunoglobulin G (IgG) (Sigma Aldrich) and 350 nm protein-A-coated silica nanospheres (Corpuscular Inc.) served as a more physiologically relevant reaction scheme. Human IgG was dissolved in deionized water and diluted to experimental concentrations (1.25, 3, 6 and 12 mg/mL). The receptor/binding solutions were driven side-by-side using a constant pressure source and the resulting fluid interface was imaged using confocal microscopy by labelling the AHA “red” with 10 ng/mL Alexa Fluor 594 and the adjacent PBS stream (containing either streptavidin, Protein A, or carboxylated nanoparticles) “green” with 10 ng/mL Alexa Fluor 488 (FIG. 14C).

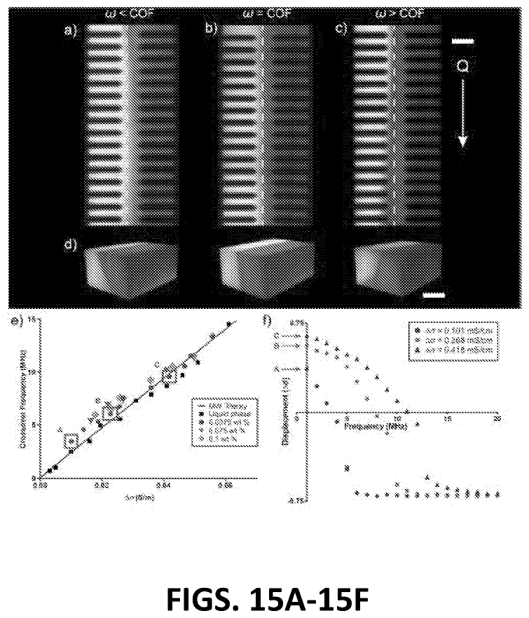

FIGS. 15A-15F illustrate graphical views of fDEP crossover frequency. When each fluid phase was driven into the main microchannel, a steep gradient in the electrical properties formed at the interface between the two fluid phases. The PBS stream had a high conductivity and a low permittivity (σ=0.35 mS/cm, ε=78.2), while the AHA stream had a low electrical conductivity and a high permittivity (σ=32 μS/cm, ε=110). When an AC electric field was applied across the microfluidic channel, the fluid interface dis-placed in a direction perpendicular to the main flow field. The direction of this displacement is sensitive to the frequency (ω) of the electric field. At low AC field frequency (ω>COF), the high conductive PBS stream displaced into the low conductive AHA stream (FIG. 15A), as the field oscillates slowly enough for mobile ions in solution to electromigrate to the interface. Because the fluid interface was engineered such that one phase had a greater electrical conductivity and the other a greater dielectric constant, the direction and magnitude of the interfacial displacement approached zero (FIG. 15B) at a characteristic crossover frequency (COF). At high AC frequency (ω>COF), however, the direction of the displacement reversed, and the high-dielectric stream displaced across the channel into the low-dielectric stream (FIG. 15C) since polarization by ionic conduction does not have enough time to occur, and dielectric polarization dominates the interfacial polarization. Con-focal micrographs depicting the 3D structure of the interface are shown in FIG. 15D. The interfacial stress as a function of frequency can be described using Maxwell-Wager polarization theory. The frequency-dependent expression for the interfacial fDEP force is

<F_{DEP}>=<σₜE>=½)[K(ω)]ε₀E₀² (3)

where Re[K(ω))] is the real (in phase) component of the Clausius-Mossotti (CM) factor which describes the degree to which the interface has polarized. The CM factor is sensitive to both differences in the conductivity and permittivity of each fluid stream (stream 1 and stream 2) in Eq. (1) where and τ=((ε2+ε1)/(σ₂+σ₁) is the charge relaxation timescale of the electrical liquid interface, which describes the characteristic timescale required for mobile ions to electromigrate to the interface. The frequency at which both dielectric and conductive charging mechanisms balance (e.g. where FDEP=0) and no displacement occurs is referred to as the COF. This is determined by setting Re[K(ω))]=0, for Eq. (2). Eq. (2) can be used to determine the COF for a microfluidic liquid interface as a function of the fluid electrical conductivity and permittivity. This expression illustrates the linear relation-ship between the interfacial electrical conductivity and the COF, as shown in FIG. 15E. The nanoparticle biosensing scheme relies on the sensitivity of the COF to the electrical properties at the microfluidic interface. If a biomolecular reaction increases the electrical conductivity difference across the interface, for example, the COF will increase. As the reaction propagates down the channel length the COF will continue to increase until the biomolecular reaction saturates. In the next section, experimental results demonstrate the ability to detect binding on nanoparticles at a liquid interface by monitoring the COF along the axial length of a microchannel.

In order to determine if the existing MW theory is appropriate to apply to a dilute colloidal suspension fDEP experiments were performed to determine the interfacial COF as a function of electrolyte conductivity with a dilute colloidal suspension of 100 nm carboxylated silica particles in PBS buffer. To obtain an accurate COF reading at the interface before diffusive mixing occurred, the COF near the entrance of the microfluidic T-junction (x*=0.2) was measured where the interface was sharp and the COF was uninfluenced by diffusive mixing. The resulting COF data is plotted against the theoretical model (Eq. (2)) in FIG. 15C for varying concentrations of colloid weight percent. The data show good agreement with the MW theory for both the pure liquid-liquid and liquid-colloid experiments. From FIG. 15C, it is therefore assumed that the colloidal suspensions used in this work are dilute enough to be mathematically and electrically approximated as a pure fluid, and that the presence of particles does not require additional modifications to the existing polarization model to accurately predict the fDEP response of the fluid interface.

Next, the net displacement of the interface is measured at different electric field frequencies and interfacial electrical conductivities in order to determine how differences in electrical conductivity across the liquid interface influence the COF and magnitude of the interfacial deflection. To accomplish this, the conductivity of the colloidal suspension was adjusted while keeping the weight percent and adjacent stream conductivity constant. In FIG. 15D the experimental displacement is plotted for three different interfacial conductivities (Δσ=0.101, 0.268 and 0.418 mS/cm). Each COF (labeled A-C) are highlighted in FIG. 15C. The data demonstrates that the magnitude of the interfacial displacement increases with interfacial conductivity at low field frequencies below the COF. However, the high frequency displacement remains unaffected since frequency regime is only influenced by dielectric differences between the two fluids.

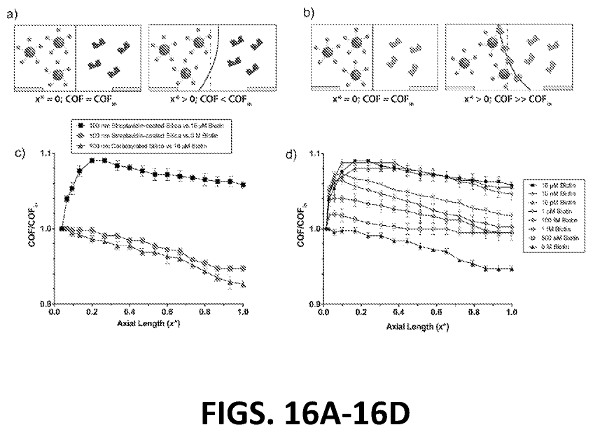

With the frequency response of a non-reacting interface known, next it was sought to determine if specific biomolecular binding on nanoparticles influences this behavior. To detect specific binding at the electrical interface, one stream was flowed with a target probe against an adjacent stream of analyte. A well-known model reaction was investigated: biotin and streptavidin. The biomolecular reaction between biotin and streptavidin are strongly associated with a very small dissociation constant, K_{d}˜10⁻¹⁵M, and therefore the reaction is rapid, nonreversible and serves as a good model system to experimentally validate the biosensing strategy. Streptavidin conjugated nanoparticles are used as a substrate for binding. With a small diffusional coefficient (10-8 cm2/s) [30], the particles provided a well-defined region of active binding sites at the liquid-liquid interface for biotin, a smaller, quickly-diffusing molecule, to rapidly diffuse towards the nanocolloidal interface and bind. First, biotin in an excess concentration (16 μM) was co-flowed against a 0.0375 wt % suspension of streptavidin-silica nanoparticles. An AC electric field was applied across the interface and the COF measured at the inlet, which is denoted here as COFᵢₙ. Next, subsequent COF measurements were performed at varying positions down the electrode array and normalized the resulting response by COFᵢₙ. Shown in FIGS. 16A-16D, the biotin-streptavidin system produced an increase in the interfacial COF with axial position. The COF continued to increase down the axial length of the channel before reaching a plateau and declining, which is indicative of a combination of binding saturation in the vicinity of the nanocolloidal-functionalized interface and diffusional smoothing between the two fluid streams. FIGS. 16A-16D illustrate schematic and graphical views of an IET bead-based bioassay.

Next, a series of control experiments were performed to validate the COF measurements. First, the same COF experiments were performed without biotin and observed that the COF does not increase, but rather it decreased over the axial channel length (FIG. 16A). This decrease is attributed to diffusional blending across the interface, where ion diffusion gradually reduces the sharp differences in electrical properties and reduces the interfacial COF. As a second non-reactive negative control, the 100 nm streptavidin-coated silica nanospheres were replaced with carboxylated silica beads of the same size and wt %, and tested the interface response against the same excess concentration of biotin (16 μM). The surface carboxyl groups (—COOH) are meant to emulate the nanocolloidal interface, while providing a non-reactive surface to observe any false positive signal from non-specific adsorption. Similar to the first negative control experiment, the COF again decreased (FIG. 16A).

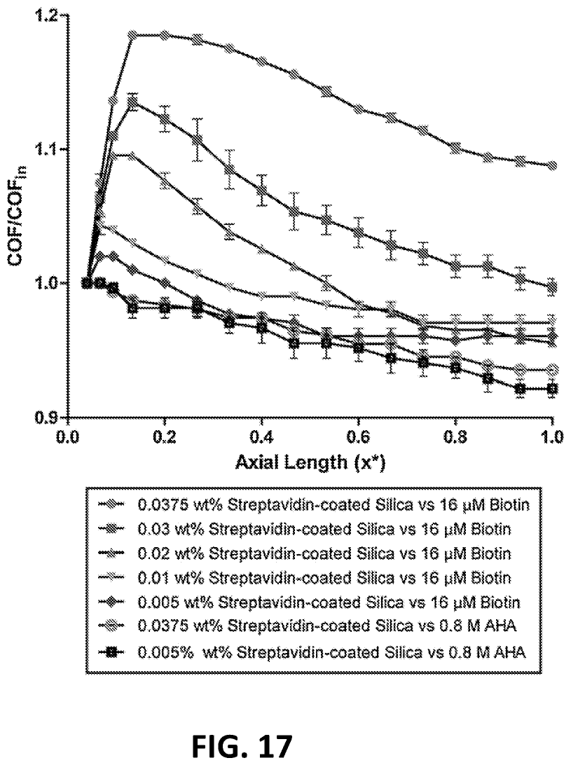

These biosensing experiments were repeated with different biotin concentrations varying several order-of-magnitude ranging between 16 μM and 500 aM (FIG. 16B). The biosensor response, as determined by the interface COF, increased with increasing concentrations of biotin (FIGS. 16A-16D). Since the wt % for each experiment was fixed at 0.0375%, the number of available binding sites remained the same for each experiment. As is expected with a substrate-based sensor response, the interface was observed to saturate for the given particle wt % at a biotin concentration of 10 pM. Above this concentration the COF signal was not influenced by further increases in the biotin concentration. Using the 3-sigma method, the experimental LOD for biotin was determined to be 500 aM. Given the dimensions of the microfluidic device (100 μm main channel width×35 μm channel height) and a total internal volume of 60 nL, this concentration translates to approximately 36 molecules of biotin in the device at any given time. FIG. 17 illustrates a graphical view of Streptavidin Limited Detection by Varying Particle wt % and a sensorgram illustrating the decrease in signal with decreasing weight percent of streptavidin-coated particles. In a related experiment, the weight percent of the nanoparticles was varied from 0.005-0.0375 wt % in order to determine if the COF is influenced by the number of available binding sites at the liquid interface. Depicted in FIG. 17, the COF response decreases with decreasing nanoparticle wt %. For this experiment, the interfacial conductivity was decreased from 0.35 mS/cm to 0.24 mS/cm. It is interesting to note that this reduction increased the contribution of the reaction relative to the baseline conductivity difference, and thus led to an increase the magnitude of the COF signal response. This increase can be observed by comparing the sensorgram data for 16 μM biotin in FIG. 17, where the maximum signal response is 1.09, to the case in FIG. 17, where the maximum response increased to 1.19. These sensor grams were compared against two different controls using carboxilated silica particles at both the low (0.005) and high (0.375) wt % concentrations. Shown in FIG. 17, the COF is not influenced by the presence of varying con-centration of non-reactive nanoparticles, and only increases when binding sites are available for biotin.

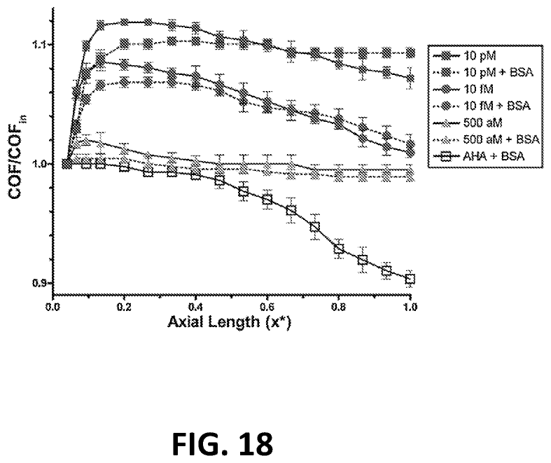

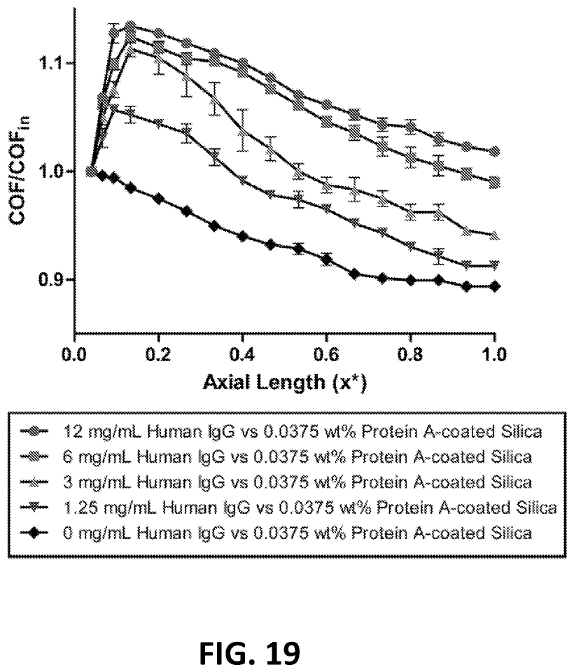

A series of experiments was performed to test the robustness and specificity of the biosensor. First, a background of 5 mg/mL bovine serum albumin (BSA) was introduced to the biotin stream. Shown in FIG. 18, the presence of BSA reduced the COF signal, but it is still capable of detecting biotin. FIG. 18 illustrates a graphical view of biotin detection in a BSA background and a sensorgram of the ability of the fDEP biosensor to detect biotin-streptavidin binding in a background of 5 mg/mL BSA. In moving toward detection of physiologically relevant targets, it was then determined if the presence of human IgG in solution using a suspension of 350 nm silica nanoparticles coated with protein A could be detected. The affinity of protein A binding to human IgG is reported to be on the order of K_{d}˜10−10M, five orders of magnitude weaker than biotin-streptavidin. In FIG. 19, human IgG was detected at concentrations as low as 1.25 mg/mL, which is comparable to relevant concentrations used in modern ELISA assays. FIG. 19 illustrates a graphical view of detection of human IgG protein A binding. The detection sensorgram shows positive detection of human IgG with 350 nm silica nanoparticles coated with protein A.

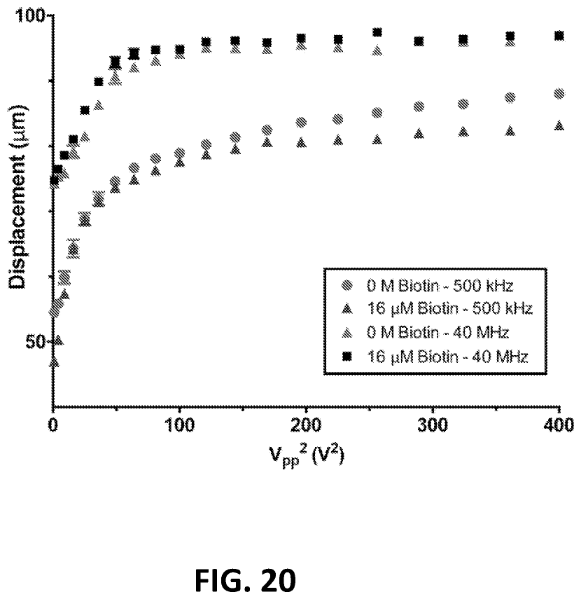

Finally, the polarization model was used to determine the electrical mechanism for why biomolecular binding increases the interface COF. It has been demonstrated that biomolecular binding in solution without nanoparticles produces a local increase in electrical conductivity difference across the liquid interface. This was demonstrated by measuring the magnitude of the interfacial deflection for different analyte concentrations during binding. This method is possible because the displacement of the interface is solely dependent on differences in electrical conductivity and permittivity below and above the COF, respectively. Therefore, by measuring the interfacial motion at both low and high frequency the electrical conductivity and the permittivity across the interface are influenced by the reaction. For reactions without nanoparticles, increased analyte concentration only produced a measurable change in the interfacial displacement at low field frequency (e.g. only influences electrical conductivity) and fDEP motion at high frequency above the COF was not influenced by binding. To determine what interfacial electrical properties were influenced by binding on nanoparticles, the magnitude of interfacial deflection was measured at varying voltages (1-20 Vₚₚ) and frequencies both below (500 kHz) and above (40 MHz) the COF for systems with and without a nanoparticle-based biomolecular reaction. In the presence of a biotin-streptavidin reaction, low frequency displacement was influenced by binding and no difference was observed at high frequency (FIG. 20). FIG. 20 illustrates a graphical view of influence of binding reaction on electrical properties measured by interface displacement.

Based on these experiments and the MW polarization model, biotin binding on streptavidin nanoparticles increases the local electrical conductivity difference across the liquid interface which produces an increase in the interfacial COF. It is worth noting that the fDEP method is unable to determine the mechanism for this conductivity increase. The negatively charged nanoparticles shed ions in their diffuse cloud during binding, which could account for the local increase in electrical conductivity.

It should be noted that the system described herein can include a computing device such as a microprocessor, hard drive, solid state drive or any other suitable computing device known to or conceivable by one of skill in the art. The computing device can be programmed with a non-transitory computer readable medium that is programmed with steps to execute the method. The computing device can receive information from the device of the present invention related to the presence and concentration of an analyte of interest. The computing device can include a display for showing the results of the diagnostic testing. Alternately, a separate microprocessor or other computing device can be included in the device of the present invention to enable detection and display of information related to the content of the sample. The computing device and/or microprocessor can receive information directly from the device for impedance spectroscopy or other means of detection.

Any such computer application will be fixed on a non-transitory computer readable medium. It should be noted that the computer application is programmed onto a non-transitory computer readable medium that can be read and executed by any of the computing devices mentioned in this application. The non-transitory computer readable medium can take any suitable form known to one of skill in the art. The non-transitory computer readable medium is understood to be any article of manufacture readable by a computer. Such non-transitory computer readable media includes, but is not limited to, magnetic media, such as floppy disk, flexible disk, hard, disk, reel-to-reel tape, cartridge tape, cassette tapes or cards, optical media such as CD-ROM, DVD, Blu-ray, writable compact discs, magneto-optical media in disc, tape, or card form, and paper media such as punch cards or paper tape. Alternately, the program for executing the method and algorithms of the present invention can reside on a remote server or other networked device. Any databases associated with the present invention can be housed on a central computing device, server(s), in cloud storage, or any other suitable means known to or conceivable by one of skill in the art. All of the information associated with the application is transmitted either wired or wirelessly over a network, via the internet, cellular telephone network, or any other suitable data transmission means known to or conceivable by one of skill in the art.

The many features and advantages of the invention are apparent from the detailed specification, and thus, it is intended by the appended claims to cover all such features and advantages of the invention, which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A non-optical, label-free microfluidic biosensor device for biomolecular detection of an analyte-of-interest in a sample comprising:
an electrical, liquid interface between two co-flowing liquids; and
a source of alternating current applied to the electrical-liquid interface in order to produce frequency-dependent liquid motion, wherein a reaction alters the frequency response.

2. The device of claim 1 wherein one of the two co-flowing liquids has a high conductivity relative to the other one of the two co-flowing liquids.

3. The device of claim 1 wherein one of the two co-flowing liquids has a high dielectric constant relative to the other one of the two co-flowing liquids.

4. The device of claim 1 wherein the analyte-of-interest is included in a stream of one of the co-flowing liquids, while a receptor for the analyte-of-interest is included in another stream of the other one of the two co-flowing liquids.

5. The device of claim 1 wherein the alternating current is applied perpendicularly across the interface causing frequency dependent liquid displacement.

6. The device of claim 1 further comprising a monitoring component configured for monitoring electrical properties of the electrical-liquid interface.

7. The device of claim 6 wherein the monitoring component configured for monitoring the electrical properties of the electrical-liquid interface is a device for impedance spectroscopy.

8. The device of claim 7 wherein the device for impedance spectroscopy is positioned downstream from the electrical-liquid interface.

9. The device of claim 1 further comprising a display related to a presence of the analyte-of-interest in the sample.

10. The device of claim 1 further comprising the analyte-of-interest comprising female hormones.

11. A method for non-optical, label-free microfluidic, biomolecular detection of an analyte-of-interest in a sample comprising:
generating an electrical, liquid interface between two co-flowing liquids; and
applying alternating current to the electrical-liquid interface to produce frequency-dependent liquid motion, wherein a reaction alters the frequency response.

12. The method of claim 11 wherein one of the two co-flowing liquids has a high conductivity relative to the other one of the two co-flowing liquids.

13. The method of claim 11 wherein one of the two co-flowing liquids has a high dielectric constant relative to the other one of the two co-flowing liquids.

14. The method of claim 11 further comprising including the analyte-of-interest is in a stream of one of the co-flowing liquids and including a receptor for the analyte-of-interest in another stream of the other one of the two co-flowing liquids.

15. The method of claim 11 further comprising applying the alternating current perpendicularly across the interface causing frequency dependent liquid displacement.

16. The method of claim 11 further comprising monitoring electrical properties of the electrical-liquid interface.

17. The method of claim 16 wherein monitoring the electrical properties of the electrical-liquid interface is done using a device for impedance spectroscopy.

18. The method of claim 17 further comprising positioning the device for impedance spectroscopy downstream from the electrical-liquid interface.

19. The method of claim 11 further comprising displaying a presence of the analyte-of-interest in the sample.

20. The method of claim 11 further comprising detecting female hormones.
